(19) United States
(12) Patent Application Publication
(10) Pub. No.: US 2012/0202822 A1
(43) Pub. Date: Aug. 9, 2012

(54) COMBINATION

(76) Inventors: Kurtis Earl Bachman, Collegeville, PA (US); Joel David Greshock, Collegeville, PA (US); Tona Gilmer, Research Triangle Park, NC (US); Sylvie Laquerre, King of Prussia, PA (US); Shannon Renae Morris, Research Triangle Park, NC (US)

(21) Appl. No.: 13/501,387

(22) PCT Filed: Oct. 12, 2010
(86) PCT No.: PCT/US10/52242

§ 371 Date: Apr. 11, 2012

Related U.S. Application Data
(60) Provisional application No. 61/250,635, filed on Oct. 12, 2009.

(51) Int. Cl. A61K 31/506; A61P 35/00

(52) U.S. Cl. 514/252.04

(57) ABSTRACT

The present invention relates to a method of treating cancer in a mammal and to pharmaceutical combinations useful in such treatment. In particular, the method relates to a novel combination comprising the MEK inhibitor: N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide, or a pharmaceutically acceptable salt or solvate thereof, and the PI3 kinase inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, pharmaceutical compositions comprising the same, and methods of using such combinations in the treatment of cancer.

### FIELD OF THE INVENTION

The present invention relates to a method of treating cancer in a mammal and to combinations useful in such treatment. In particular, the method relates to a novel combination comprising the B-Raf inhibitor: N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, pharmaceutical compositions comprising the same, and methods of using such combinations in the treatment of cancer.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP—Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity including benign and malignant proliferative disorders as well as diseases resulting from inappropriate activation of the immune and nervous systems. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The protein serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases as well.

Mitogen-activated protein kinase (MAPK) Kinase/extracellular signal-regulated kinase (ERK) kinase (hereinafter referred to as MEK) is known to be involved in the regulation of numerous cellular processes. The Raf family (B-Raf, C-Raf etc.) activates the MEK family (MEK-1, MEK-2 etc.) and the MEK family activates the ERK family (ERK-1 and ERK-2). Broadly, the signaling activity of the RAF/MEK/ERK pathway controls mRNA translation. This includes genes related to the cell cycle. Hence, hyperactivation of this pathway can lead to uncontrolled cell proliferation. Deregulation of the RAF/MEK/ERK pathway by ERK hyperactivation is seen in approximately 30% of all human malignancies (Allen, L F, et al. Semin. Oncol. 2003. 30(5 Suppl 16):105-16). Activating BRAF mutations have been identified at a high frequency in specific tumor types (e.g., melanomas) (Davies, H. et al. Nature. 2002. 417:949-54). Approximately 90% of all identified BRAF mutations that occur in human cancer are a T1799 transversion mutations in exon 15, which results in a V600 E/D/K(T1799A) amino acid substitution (Wellbrock, C. et al. Nat. Rev. Mol. Cell. Biol. 2004. 5:875-85; Wan, P T et al. Cell. 2004. 116:855-67). This mutation appears to mimic regulatory phosphorylation and increases BRAF activity approximately 10-fold compared to wild type (Davies, H. et al. Nature. 2002. 417:949-54). The frequency of this activating mutation and the pathway addiction to which it leads makes mutated BRAF an extremely attractive target.

The phosphoinositide 3-kinase (PI3K) pathway is among the most commonly activated pathways in human cancer. The function and importance of this pathway in tumorigenesis and tumor progression is well established (Samuels & Ericson. Curr. Opp in Oncology, 2006. 18: 77-82). PI3K-AKT signaling appears to be a pivotal modulator of cell survival, proliferation and metabolism. This includes the activation of mammalian target of rapamycin (mTOR), a PI3K protein family member and direct regulator of cell growth and translation. Thus, the deregulation of PI3K/AKT/mTOR signaling in tumors contributes to a cellular phenotype that demonstrates numerous hallmarks of malignancies, which includes unlimited reproductive potential and the evasion of apoptosis (Hanahan & Weinberg, Cell. 2000. 100:57-70).

The PI3K family consists of 15 proteins that share sequence homology, particularly within their kinase domains; however; they have distinct substrate specificities and modes of regulation (Vivanco & Sawyers. Nat. Rev. Cancer, 2002.2:489-501). Class I PI3-kinases phosphorylate inositol-containing lipids, known as phosphatidylinositols (PtdIns) at the 3 position. The primary substrate of Class I family members, PtdIns-4,5-P2 (PIP2) is converted to PtdIns-3,4,5-P3 (PIP3) by these kinases. PIP3 is a critical second messenger which recruits proteins that contain pleckstrin homology domains to the cell membrane where they are activated. The most studied of these proteins is AKT which promotes cell survival, growth, and proliferation. Upon activation, AKT moves to the cytoplasm and nucleus where it phosphorylates numerous substrates, including mTOR (TORC1). In addition to AKT, PI3K activates other pathways that are implicated in carcinogenesis such as PDK1, CDC42 and RAC1 (Samuels & Ericson. Curr. Opp in Oncology, 2006. 18: 77-82).

In the study of human tumors, activation of the PI3K/AKT/mTOR signaling pathway can occur via numerous mechanisms. Genetic deregulation of the pathway is common and can occur in a number of ways (reviewed in Samuels & Ericson. Curr. Opp in Oncology, 2006. 18: 77-82). Activating mutations of the PIK3CA gene (coding for the p110α catalytic subunit of PI3K) occur in a significant percentage of human tumors including breast, ovarian, endometrial, and colorectal cancer. Activating DNA amplifications of this gene also occur less frequently in a number of different tumor types. Mutations in the p85α regulatory subunit of PI3K (PIK3R1), which are thought to disrupt the C2-iSH2 interaction between PIK3R1 and PIK3CA, occur in ovarian, glioblastoma and colorectal cancer. The tumor suppressor PTEN, which dephosphorylates PIP3 to generate PIP2 and thus acts as an inhibitor of the PI3K pathway, is commonly mutated, deleted, or epigenetically silenced. Finally, the pathway can also be genetically activated downstream of PI3K by DNA amplification or mutation of AKT; however these genetic events occur much less frequently in human cancer. Inhibiting PI3K isoforms, particularly PI3Kα, are known to be useful in the treatment of cancer (see for example WO 05/121142, WO 08/144,463, WO 08/144,464, WO 07/136,940).

It would be useful to provide a novel, mono or combination therapy which provides more effective and/or enhanced treatment of an individual suffering the effects of cancer.

### SUMMARY OF THE INVENTION

One embodiment of this invention provides a combination comprising:

(i) a compound of Structure (I):

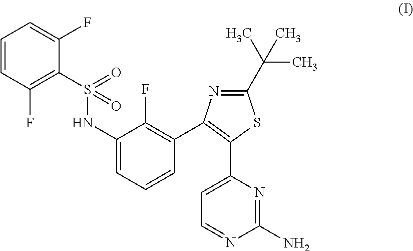

N-{3-[5-(2-amino-4-pyrimidinyl)-2-(1,1-dimethylethyl)-1,3-thiazol-4-yl]-2-fluorophenyl}-2,6-difluorobenzenesulfonamide, (hereinafter Compound A)
   or a pharmaceutically acceptable salt thereof; and
   (ii) a compound of Structure (II):

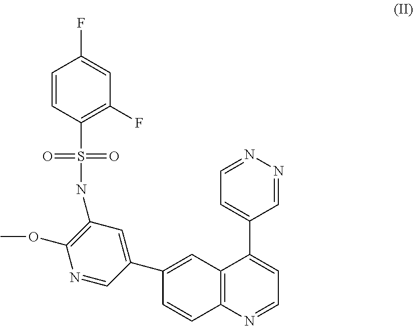

2,4-difluoro-N-{2-(methyloxy)-5-[4-(4-pyridazinyl)-6-quinolinyl]-3-pyridinyl}benzenesulfonamide (hereinafter Compound B)
   or a pharmaceutically acceptable salt thereof.

One embodiment of this invention provides a method of treating cancer in a human in need thereof which comprises the in vivo administration of a therapeutically effective amount of a combination of Compound A, or a pharmaceutically acceptable salt or solvate thereof, and Compound B, or a pharmaceutically acceptable salt thereof, to such human.

One embodiment of this invention provides a method of treating cancer in a human in need thereof which comprises the in vivo administration of a therapeutically effective amount of a combination of Compound A, or a pharmaceutically acceptable salt or solvate thereof, and Compound B, or a pharmaceutically acceptable salt thereof, to such human,
wherein the combination is administered within a specified period, and
wherein the combination is administered for a duration of time.

One embodiment of this invention provides a method of treating cancer in a human in need thereof which comprises the in vivo administration of a therapeutically effective amount of a combination of Compound A, or a pharmaceutically acceptable salt or solvate thereof, and Compound B, or a pharmaceutically acceptable salt thereof, to such human,
Wherein compounds A and B are administered sequentially.

### DETAILED DESCRIPTION OF THE INVENTION

The present invention relates to combinations that exhibit antiproliferative activity. Suitably, the method relates to methods of treating cancer by the co-administration of N-{3-[5-(2-amino-4-pyrimidinyl)-2-(1,1-dimethylethyl)-1,3-thiazol-4-yl]-2-fluorophenyl}-2,6-difluorobenzenesulfonamide, (Compound A), or a pharmaceutically acceptable salt or solvate, suitably the dimethyl sulfoxide solvate thereof, which compound is represented by Structure I:

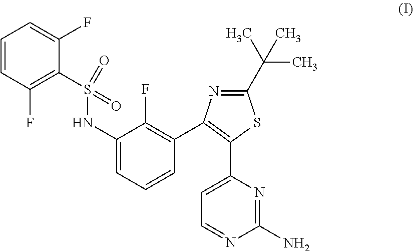

and 2,4-difluoro-N-{2-(methyloxy)-5-[4-(4-pyridazinyl)-6-quinolinyl]-3-pyridinyl}benzenesulfonamide (Compound B), or a pharmaceutically acceptable salt thereof; which compound is represented by the following structure

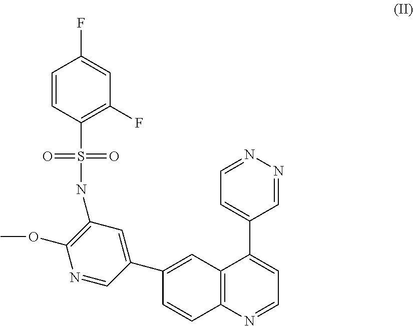

Compound A is disclosed and claimed, along with pharmaceutically acceptable salts thereof, as being useful as an inhibitor of B-Raf activity, particularly in treatment of cancer, in International Application No. PCT/US2009/042682, having an International filing date of May 4, 2009, International Publication Number WO 2009/137391 and an International Publication date of WO 2009/137391, the entire disclosure of which is hereby incorporated by reference, Compound A is the compound of Example 58. Compound A can be prepared as described in International Application No. PCT/US2009/042682.

Suitably, Compound A is in the form of a methanesulfonate salt. This salt form can be prepared by one of skill in the art from the description in International Application No. PCT/US2009/042682, having an International filing date of May 4, 2009.

Compound B is disclosed and claimed, along with pharmaceutically acceptable salts thereof, as being useful as an inhibitor of PI3K activity, particularly in treatment of cancer, in International Application No. PCT/US2008/063819, having an International filing date of May 16, 2008; International Publication Number WO 2008/1444463 and an International Publication date of Nov. 27, 2008, the entire disclosure of which is hereby incorporated by reference, Compound B is the compound of example 345. Compound B can be prepared as described in International Application No. PCT/US2008/063819.

Suitably, Compound B is in the form of free base.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

The pharmaceutically acceptable salts of the compounds of the invention are readily prepared by those of skill in the art.

Also, contemplated herein is a method of treating cancer using a combination of the invention where Compound A, or a pharmaceutically acceptable salt or solvate thereof, and/or Compound B or a pharmaceutically acceptable salt thereof are administered as pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “periodically administration” or variations thereof, is meant that the drug is administered to the human with drug holidays. A drug holiday (sometimes also called a drug vacation, medication vacation, structured treatment interruption or strategic treatment interruption) is when a patient stops taking a medication(s) for a period of time; anywhere from a few days to several months

By the term “combination” and derivatives thereof, as used herein is meant either simultaneous administration or any manner of separate sequential administration of a therapeutically effective amount of Compound A, or a pharmaceutically acceptable salt or solvate thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt or solvate thereof, and Compound B, or a pharmaceutically acceptable salt thereof, according to the invention. When both compounds are administered simultaneously, the combination kit can contain Compound A, or a pharmaceutically acceptable salt or solvate thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable salt or solvate thereof, and Compound B, or a pharmaceutically acceptable salt thereof, in separate pharmaceutical compositions. The combination kit can comprise Compound A, or a pharmaceutically acceptable salt or solvate thereof, and Compound B, or a pharmaceutically acceptable salt thereof, in separate pharmaceutical compositions in a single package or in separate pharmaceutical compositions in separate packages.

In one aspect there is provided a combination kit comprising the components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier.

In one embodiment of the invention the combination kit comprises the following components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier,
   wherein the components are provided in a form which is suitable for sequential, separate and/or simultaneous administration.

In one embodiment the combination kit comprises:
a first container comprising Compound A, or a pharmaceutically acceptable salt or solvate thereof, in association with a pharmaceutically acceptable carrier; and
a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

Unless otherwise defined, in all dosing protocols described herein, the regimen of compounds administered does not have to commence with the start of treatment and terminate with the end of treatment, it is only required that the number of consecutive days in which both compounds are administered and the optional number of consecutive days in which only one of the component compounds is administered, or the indicated dosing protocol—including the amount of compound administered, occur at some point during the course of treatment.

As used herein the term “Compound A²” means ---Compound A, or a pharmaceutically acceptable salt or solvate thereof---.

As used herein the term “Compound B²” means ---Compound B, or a pharmaceutically acceptable salt thereof---.

Suitably the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. When both compounds of the invention are administered once a day the specified period refers to administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered for an indicated number of consecutive days.

Regarding “specified period” administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and derivates thereof, as used herein is meant that one of Compound A² and Compound B² is administered once a day for one or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding sequential administration:

Suitably, one of Compound A² and Compound B² is administered for from 2 to 30 consecutive days, followed by an optional drug holiday, followed by administration of the other of Compound A² and Compound B² for from 2 to 30 consecutive days. Suitably, one of Compound A² and Compound B² is administered for from 2 to 21 consecutive days, followed by an optional drug holiday, followed by administration of the other of Compound A² and Compound B² for from 2 to 21 consecutive days. Suitably, one of Compound A² and Compound B² is administered for from 2 to 14 consecutive days, followed by a drug holiday of from 1 to 14 days, followed by administration of the other of Compound A² and Compound B² for from 2 to 14 consecutive days. Suitably, one of Compound A² and Compound B² is administered for from 3 to 7 consecutive days, followed by a drug holiday of from 3 to 10 days, followed by administration of the other of Compound A² and Compound B² for from 3 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 10 mg to about 300 mg; suitably, the amount will be selected from about 30 mg to about 280 mg; suitably, the amount will be selected from about 40 mg to about 260 mg; suitably, the amount will be selected from about 60 mg to about 240 mg; suitably, the amount will be selected from about 80 mg to about 220 mg; suitably, the amount will be selected from about 90 mg to about 210 mg; suitably, the amount will be selected from about 100 mg to about 200 mg, suitably, the amount will be selected from about 110 mg to about 190 mg, suitably, the amount will be selected from about 120 mg to about 180 mg, suitably, the amount will be selected from about 130 mg to about 170 mg, suitably, the amount will be selected from about 140 mg to about 160 mg, suitably, the amount will be 150 mg. Accordingly, the amount of Compound A² administered as part of the combination according to the present invention will be an amount selected from about 10 mg to about 300 mg. For example, the amount of Compound A² administered as part of the combination according to the present invention is suitably selected from 10 mg, 20 mg, 30 mg, 40 mg, 50 mg, 60 mg, 70 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg and 300 mg. Suitably, the selected amount of Compound A² is administered from 1 to 4 times a day. Suitably, the selected amount of Compound A² is administered twice a day. Suitably, the selected amount of Compound A² is administered once a day. Suitably, the administration of Compound A² will begin as a loading dose. Suitably, the loading dose will be an amount from 2 to 100 times the maintenance dose; suitably from 2 to 10 times; suitably from 2 to 5 times; suitably 2 times; suitably 3 times; suitably 4 times; suitably 5 times. Suitably, the loading does will be administered from 1 to 7 days; suitably from 1 to 5 days; suitably from 1 to 3 days; suitably for 1 day; suitably for 2 days; suitably for 3 days, followed by a maintenance dosing protocol.

Suitably, the amount of Compound B² administered as part of the combination according to the present invention will be an amount selected from about 0.25 mg to about 75 mg; suitably, the amount will be selected from about 0.5 mg to about 50 mg; suitably, the amount will be selected from about 1 mg to about 25 mg; suitably, the amount will be selected from about 2 mg to about 20 mg; suitably, the amount will be selected from about 4 mg to about 16 mg; suitably, the amount will be selected from about 6 mg to about 12 mg; suitably, the amount will be about 10 mg. Accordingly, the amount of Compound B² administered as part of the combination according to the present invention will be an amount selected from about 0.5 mg to about 50 mg. For example, the amount of Compound B² administered as part of the combination according to the present invention can be 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 20 mg, 21 mg, 22 mg, 23 mg, 25 mg, 26 mg, 27 mg, 28 mg, 29 mg, 30 mg, 35 mg, 40 mg, 45 mg, or 50 mg.

As used herein, all amounts specified for Compound A² and Compound B² are indicated as the administered amount of free or unsalted and unsolvated compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The combinations of the present invention are tested for efficacy, advantageous and synergistic properties according to known procedures. Suitably, the combinations of the invention are tested for efficacy, advantageous and synergistic properties generally according to the following combination cell proliferation assays. Cells are plated in 96 or 384-well plates in culture media appropriate for each cell type, supplemented with 10% FBS and 1% penicillin/streptomycin, and incubated overnight at 37° C., 5% CO₂. Cells are treated in a grid manner with dilution of Compound A² (10 dilutions, including no compound, of 3-fold dilutions starting from 0.50-10 μM) and also treated with Compound B² (10 dilutions, including no compound, of 3-fold dilutions starting from 0.10 μM) and incubated as above for a further 72 hours. In some instances compounds are added in a staggered manner and incubation time can be extended up to 7 days. Cell growth is measured using CellTiter-Glo® reagent according to the manufacturer's protocol and signals are read on a PerkinElmer EnVision™ reader set for luminescence mode with a 0.5-second read. Data are analyzed as described below.

Results are expressed as a percentage of the t=0 value and plotted against compound(s) concentration. The t=0 value is normalized to 100% and represents the number of cells present at the time of compound addition. The cellular response is determined for each compound and/or compound combination using a 4parameter curve fit of cell viability against concentration using the IDBS XLfit plug-in for Microsoft Excel software and determining the concentration required for 50% inhibition of cell growth (gIC₅₀). Background correction is made by subtraction of values from wells containing no cells. For each drug combination a Combination Index (CI), Excess Over Highest Single Agent (EOHSA) and Excess Over Bliss (EOBliss) are calculated according to known methods such as described in Chou and Talalay (1984) Advances in Enzyme Regulation, 22, 37 to 55; and Berenbaum, M C (1981) Adv. Cancer Research, 35, 269-335.

Because the combinations of the present invention are active in the above assays they exhibit advantageous therapeutic utility in treating cancer.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method for treating or lessening the severity of a cancer selected from lung, pancreatic, and colon.

Suitably, the present invention relates to a method of treating or lessening the severity of a cancer that is either wild type or mutant for certain biomarker(s).

Suitably, the present invention relates to a method of treating or lessening the severity of a cancer that is either wild type or mutant for Raf and either wild type or mutant for PI3K/Pten. This includes patients wild type for both Raf and PI3K/PTEN, mutant for both Raf and PI3K/PTEN, mutant for Raf and wild type for PI3K/PTEN and wild type for Raf and mutant for PI3K/PTEN.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

Cancers that are either wild type or mutant for biomarker(s) and either wild type or mutant for PI3K/Pten are identified by known methods.

For example, wild type or mutant Ras/Raf or PI3K/PTEN tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry. Suitably, Pyrophosphorolysis-activated polymerization (PAP) and/or PCR methods may be used. Liu, Q et al; Human Mutation 23:426-436 (2004).

This invention provides a combination comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for a combination comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, for use in therapy.

This invention also provides for a combination comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, for use in treating cancer.

This invention also provides a pharmaceutical composition comprising a combination of N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides a combination kit comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for the use of a combination comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament.

This invention also provides for the use of a combination comprising N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament to treat cancer.

This invention also provides a method of treating cancer which comprises administering a combination of N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, to a subject in need thereof.

The following examples are intended for illustration only and are not intended to limit the scope of the invention in any way.

### Experimental Details

N-{3-[5-(2-amino-4-pyrimidinyl)-2-(1,1-dimethylethyl)-1,3-thiazol-4-yl]-2-fluorophenyl}-2,6-difluorobenzenesulfonamide (Compound A) is disclosed and claimed, along with pharmaceutically acceptable salts thereof, as being useful as an inhibitor of B-Raf activity, particularly in treatment of cancer, in International Application No. PCT/US2009/042682, having an International filing date of May 4, 2009, International Publication Number WO 2009/137391 and an International Publication date of WO 2009/137391, the entire disclosure of which is hereby incorporated by reference, Compound A is the compound of Example 58. Compound A can be prepared as described in International Application No. PCT/US2009/042682.

PI3K inhibitors which are suitable for use in the present combinations, particularly 2,4-difluoro-N-{2-(methyloxy)-5-[4-(4-pyridazinyl)-6-quinolinyl]-3-pyridinyl}benzenesulfonamide

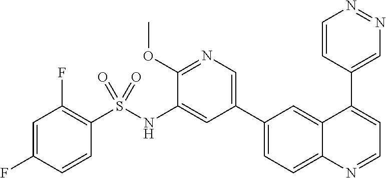

can be prepared according to International Patent Publication No. WO08/144,463 (Example 345)
Study #1: In Vitro Cell Growth Inhibition and Apoptosis Induction by Compound A, Compound B and their Combination in Tumor Cell Lines

### Colon Cancer Cell Lines

### Experimental Preparation(s)

Combination drug tests with Compounds A and B were conducted using a panel of cell lines from human colon cancers (n=25) (Table 1). Cell lines were purchased commercially [from ATCC (Manassas, Va., USA) or DSMZ (Braunschweig, Germany)] and grown in RPMI-1640 supplemented with 2 mM glutamine, 1 mM sodium pyruvate and 10% fetal bovine serum and maintained at 37° C. and 5% CO₂ in a humid incubator.

### Experimental Protocol(s)

### Fixed Ratio Drug Combination Assay

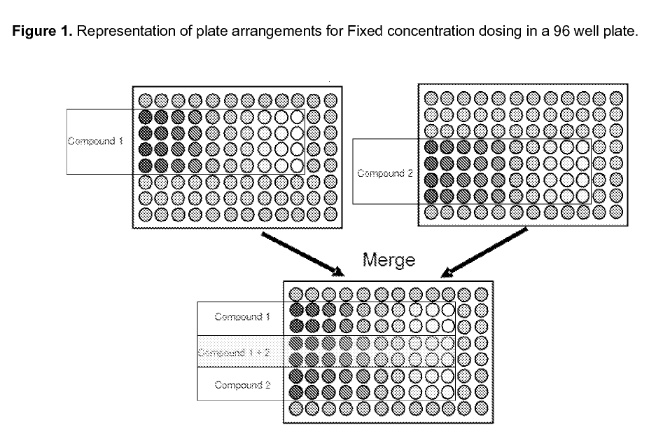

The dilution design of the Fixed Ratio Drug Combination Assay can be seen in FIG. 1. First, the test compounds were prepared as 10 mM stocks in 100% dimethyl sulfoxide (DMSO). Further dilutions of the compounds were made with DMSO. The first test compound (designated as Compound A) is diluted horizontally in a 96 well microtiter plate in rows B-E using a 3-fold dilution series for 10 dilution points. A second test compound (designated as Compound B) is diluted horizontally in a separate 96 well microtiter plate in rows D-G using a 3-fold dilution series for 10 dilution points. The two compounds are combined using equal volumes from each drug plate into cell culture media. This results in a 1:50 dilution of the drugs in the cell culture media. Compound A is individually titrated in rows B and C, while only Compound B is dosed in rows F and G of the plate. An additional 1:10 dilution of the drugs is performed in cell culture media prior to addition to the cells. Drug addition to the cells results in a further 1:2 dilution of drugs. The total dilution of the drug plate to the cells is 1:1000. The final dosing concentration range for Compound B was 0.1-1000.0 nM and was 0.5-10000.0 nM for Compound A. The positive control consists of culture media with DMSO at 0.1% and cells and no drug. The negative control consists of culture media with DMSO at 0.1%. solution.

Assays were performed in 96 well microtiter plates with appropriate seeding densities estimated from previous studies of each cell line. Following dosing, the cell lines are incubated at 37° C., 5% CO₂ in humid air for 72 hours. Cell proliferation was measured using the CellTiter Glo (Promega Corporation, Madison, Wis., USA) reagent according to the manufacturer's protocol. The plates are treated with CellTiter Glo solution and are analyzed for RLU (relative light units) using a Molecular Devices SpectraMax M5 (Sunnyvale, Calif., USA) plate reader.

### Data Analysis

Results are expressed as a percentage of the number of cells present at the time of compound addition value (T₀) and plotted against compound(s) concentration. The percent intensity values were used in model 205 of the IDBS XLfit plug-in for Microsoft Excel to calculate gIC₅₀ values using a 4 parameter logistical fit. Background correction is made by subtraction of values from wells containing no cells. The midpoint of the growth window (the gIC₅₀) falls half way between the number of cells at the time of compound addition and the growth of control cells treated with DMSO at 72 hrs. The number of cells at time zero is divided from the intensity value at the bottom of the response curve (Yₘᵢₙ) to generate a measure for cell death (Yₘᵢₙ/T₀). A value below 1 for Yₘᵢₙ/T₀ indicates stronger potency with the treatment when compared to higher values. For duplicate assays, all response metrics are averaged for presentation.

Three independent metrics were used to analyze the combinatorial effects on growth inhibition of Compound B and Compound A.

### Data Analysis

Three independent metrics were used to analyze the combinatorial effects on growth inhibition of Compound B and Compound A.
1. Excess over Highest Single Agent (EOHSA)—One standard criterion for measuring drug combinatorial effects is analyzing the effects on cell growth inhibition in absolute terms. In this case, the combination of drugs is compared to the more responsive of the two individual treatments (single agent). For each combination experiment, the percent effect relative to the highest single agent for each dose along the curve is generated. This measure of “Excess of Highest Single Agent (EOHSA)” is one of the criteria used for evaluating synergy of drug combinations. (Borisy A A Elliott P J, Hurst N W, Lee M S, Lehar J, Price E R, Serbedzija G, Zimmermann G R, Foley M A, Stockwell B R, Keith C T. Systematic discovery of multicomponent therapeutics. Proc Natl Acad Sci USA. 2003 Jun. 24; 100(13):7977-82)
2. Bliss synergy—A second criterion often used to determine combination synergy is evaluating the excess inhibition over Bliss independence or “additivity” (Bliss, C. I, Mexico, D F, The Toxicity of Poisons Applied Jointly. Annals of Applied Biology 1939, Vol 26, Issue 3, August 1939). The model assumes a combined response of the two compounds independently using the following:

Score=Eₐ+E_{b}−(Eₐ*E_{b})
Where Eₐ is the effect (or percent inhibition) of compound A and E_{b} is the effect of compound B. The resulting effect of the combination of the two compounds is compared to their predicted additivity by Bliss and a synergy score is generated for each dose along the response curve.
3. Combination Index (CI)— A third criterion for evaluation of synergy is Combination Index (CI) derived from the Chou and Talalay (Chou T C, Talalay P. Quantitative analysis of dose-effect relationships: the combined effects of multiple drugs or enzyme inhibitors. Adv Enzyme Regul. 1984; 22:27-55). The following equation is a model used for compounds that behave with different mechanisms of action (mutually non-exclusive formula).

$$Combination Index = \frac{{D}_{a} in a : b}{{IC}_{50 ( a )}} + \frac{{D}_{b} in a : b}{{IC}_{50 ( b )}} + \frac{( {D}_{a} in a : b ) ( {D}_{b} in a : b )}{( {IC}_{50 ( a )} ) ( {IC}_{50 ( b )} )}$$

The lower the CI the more synergy the combination potentially has. A CI greater than 1 suggests that the combination being studied may be antagonistic. CI scores are also generated for inhibitory concentrations of 25% (IC₂₅) and 75% (IC₇₅) by replacing the IC₅₀ in the formula above for each compound with the respective inhibitory concentration.

The percent intensity values were used in model 205 of XLfit in Microsoft Excel to calculate gIC₅₀ values using a 4 parameter logistical fit. The midpoint of the growth window (the gIC₅₀) falls half way between the number of cells at the time of compound addition (T=0) and the growth of control cells treated with DMSO at 72 hrs. The number of cells at time zero (T₀) is divided from the intensity value at the bottom of the response curve (Yₘᵢₙ) to generate a measure for cell death (Yₘᵢₙ/T₀). A value below 1 for Yₘᵢₙ/T₀ indicates stronger potency with the treatment when compared to higher values.

For EOHSA and Bliss, a synergy score must be seen in both technical replications within an experiment to make an appropriate designation (synergy, modest synergy, etc). Each combination experiment contains a replicate for the two compounds as single agents as well as a technical replicate for the combination.

Synergy scores for EOHSA and Bliss, at extremely low concentrations, (e.g. Dose 1, dose 2) are subject to higher variation and generally excluded from the analysis. Conversely, synergy scores at the highest concentrations (Dose 10), far outside of the therapeutic dosing range, are generally excluded from analysis since the effects observed are more susceptible to off-target events.

For EOHSA and Bliss Synergy measures, a score is generated for each dose along the response curve. Scores were categorized as being ‘Antagonistic’ (<−10), ‘Additive’ (−10-10), ‘Modest Synergy’ (10-20) or ‘Synergistic’ (>20). These scores reflect the percentage over the highest agent or percentage greater than Bliss additivity, depending on which model is being interpreted.

For the Combination Index, the lower the CI, the more synergy the combination potentially has. Scores between 0 and 0.7 were considered to be synergistic, while scores between 0.7 and 0.9 were considered to be modest synergy. All other scores did not indicate synergy for the Combination index.

For those cell lines that never reached an inhibitory concentration of 25% for 1 of the compounds in the combination, a CI value cannot be calculated and ‘NA’ was listed for the CI.

### Cell Line Mutation Data

Point mutation data was collated for the status for the KRAS, BRAF, PIK3CA and PTEN genes. The data source is the cancer cell line mutation screening data published as part of the Catolog of Somatic Mutations in Cancer database (COSMIC) (Bamford S. et al. Br. J. Cancer. 2004. 91:355-58). In order to ensure that the identity of the cell lines used in the proliferation assay matched that in the COSMIC database, a genotype comparison was done between those cell lines in the sensitivity screen and those in COSMIC. Specifically, this entailed:
1. Calculating the genotypes for each cell line using the Affymetrix 500K ‘SNP Chip’ (Affymetrix, Inc., Sunnyvale, Calif.) and the RLMM algorithm (Rabbee & Speed, Bioinformatics, 2006. 22: 7-12).
2. Identifying the genotype matches of each cell line to those pre-calculated for each cell line having mutation profiles in COSMIC.
3. Assigning mutation status for each cell line in based upon the genotype matches.

### Results

All genes were mutated in a subset of the cell lines. Gene mutations of KRAS were found in 60% (15/25) of samples, while PIK3CA was mutated in 40% (10/25), BRAF in 20% (5/25), and PTEN in 4% (1/25) of cell lines (data found in Table 1).
A comprehensive categorization of the degree of synergy was done for each cell line treated with the combination of the PI3K inhibitor Compound B and BRAF inhibitor Compound A. Notably, cell line SW1116 was excluded from analyses due to low data quality. Cell lines were considered to have synergy when at least one metric was scored as synergistic. By this criteria, 54% (13/24) of cell lines showed synergy. The Yₘᵢₙ/Tₒ ratios, where values <1 show higher cell net cell death compared to higher values, were decreased in 79% (19/24) of cell lines compared to the most cytotoxic single agent. The combined dosing of Compounds A & B yielded Yₘᵢₙ/T₀ ratios <1 in 71% (17/24) of cell lines. Synergy and cytotoxicity data for colon cancer cell lines is presented in Table 2.

TABLE 1
Scores Panel of colon cancer cell lines used in combination studies.

| Cell Line | Diagnosis/Histology | KRAS | PIK3CA | BRAF | PTEN |
|---|---|---|---|---|---|
| HT29 | Carcinoma | WT | 1345C > A | 1799T > A | WT |
| SW948 | Adenocarcinoma | 182A > T | 1624G > A | WT | WT |
| T84 | Carcinoma | 38G > A | 1624G > A | WT | WT |
| HCT15 | Adenocarcinoma | 38G > A | 1633G > A | WT | WT |
| HCT8 | Adenocarcinoma | 38G > A | 1633G > A | WT | WT |
| DLD1 | Carcinoma | 38G > A | 1633G > A | WT | WT |
| NCIH508 | Adenocarcinoma | WT | 1633G > A | 1786G > C | WT |
| LS174T | Adenocarcinoma | 35G > A | 3140A > G | WT | WT |
| HCT116 | Carcinoma | 38G > A | 3140A > G | WT | WT |
| RKO | Carcinoma | WT | 3140A > G | 1799T > A | WT |
| SW1463 | Carcinoma | 34G > T | WT | WT | WT |
| SW837 | Adenocarcinoma | 34G > T | WT | WT | WT |
| SW1116 | Carcinoma | 35G > C | WT | WT | WT |
| SW480 | Adenocarcinoma | 35G > T | WT | WT | WT |
| SW403 | Carcinoma | 35G > T | WT | WT | WT |
| NCIH747 | Adenocarcinoma | 38G > A | WT | WT | WT |
| LS1034 | Adenocarcinoma | 436G > A | WT | WT | WT |
| HCC2998 | Carcinoma | 436G > A | WT | WT | WT |
| NCIH716 | Adenocarcinoma | WT | WT | WT | WT |
| NCIH630 | Adenocarcinoma | WT | WT | WT | WT |
| KM12 | Adenacarcinoma | WT | WT | WT | 800delA, |
| | | | | | 385G > T |
| SW48 | Adenocarcinoma | WT | WT | WT | WT |
| COLO320DM | Adenocarcinoma | WT | WT | WT | WT |
| COLO205 | Adenocarcinoma | WT | WT | 1799T > A | WT |
| SW1417 | Adenacarcinoma | WT | WT | 1799T > A | WT |

Table 1 key
Cell Line = Cell line name
Diagnosis/Histology = Pathological diagnosis of tissue
KRAS/BRAF/PIK3CA/PTEN = Mutation status; WT = Wild Type

TABLE 2
Basic measures and Synergy calls for each of the Colon cell lines.

| | Compound A | | Compound B | | Combination | | Synergy Metrics | | |
|---|---|---|---|---|---|---|---|---|---|
| | gIC₅₀ | | gIC₅₀ | | gIC₅₀ | | | | Comb |
| Cell Line | (nM) | Yₘᵢₙ/T₀ | (nM) | Yₘᵢₙ/T₀ | (nM) | Yₘᵢₙ/T₀ | EOHSA | BLISS | Index |
| COLO205 | 7.3 | 1.2 | 12.0 | 1.4 | 2.7 | <0.1 | Modest | No Synergy | Synergy |
| COLO320DM | >10000 | 4.9 | 4.7 | 1.5 | 8.2 | 1.2 | Modest | No Synergy | Synergy |
| DLD1 | 198.6 | 6.7 | 20.5 | 1.8 | 66.4 | 1.1 | Synergy | No Synergy | Synergy |
| HCC2998 | 2396.1 | 2.8 | 4.7 | 0.9 | 19.3 | 0.7 | Modest | No Synergy | No |
| | | | | | | | | | Synergy |
| HCT116 | >10000 | 9.2 | 34.2 | 4.1 | 94.2 | 1.3 | Synergy | No Synergy | Synergy |
| HCT15 | >10000 | 6.1 | 7.2 | 2.4 | 48.9 | 1.3 | No Synergy | Modest | |
| HCT8 | >10000 | 9.2 | 8.4 | 1.6 | 42.9 | 1.3 | Modest | Modest | |
| HT29 | 17.2 | 4.0 | 7.4 | 2.0 | 4.0 | 0.8 | Modest | Modest | Synergy |
| KM12 | >10000 | 8.8 | 22.1 | 1.4 | 373.4 | 1.7 | No Synergy | No Synergy | |
| LS1034 | >10000 | 1.9 | 1.4 | 0.5 | 3.6 | 0.5 | Modest | No Synergy | |
| LS174T | >10000 | 7.0 | 34.1 | 0.8 | 332.9 | 0.8 | Mixed | Synergy | |
| NCIH508 | 365.6 | 2.1 | 1.2 | 0.5 | 4.2 | 0.4 | Modest | Modest | Synergy |
| NCIH630 | 5000.0 | 1.9 | 18.3 | 0.9 | 2.4 | 0.7 | Synergy | Synergy | |
| NCIH716 | >10000 | 1.3 | 1.1 | 0.7 | 0.1 | 0.5 | Modest | Modest | Synergy |
| NCIH747 | >10000 | 1.3 | 2.7 | 0.5 | 0.2 | 0.2 | Synergy | Synergy | Synergy |
| RKO | 8951.3 | 6.7 | 10.9 | 3.0 | 15.1 | 0.3 | Synergy | Synergy | |
| SW1417 | 245.6 | 1.1 | 1.4 | 1.3 | 0.6 | 0.6 | Synergy | Modest | Synergy |
| SW1463 | >10000 | 1.9 | 8.2 | 0.9 | 142.5 | 0.5 | Modest | Synergy | |
| SW403 | >10000 | 2.7 | 1.5 | 0.5 | 7.4 | 0.5 | No Synergy | No Synergy | |
| SW48 | >10000 | 4.7 | 2.6 | 0.4 | 29.4 | 0.7 | Modest | Modest | |
| SW480 | >10000 | 2.9 | 5.4 | 1.1 | 93.5 | 1.1 | Synergy | Modest | |
| SW837 | >10000 | 2.3 | 6.4 | 0.8 | 8.3 | 0.5 | Modest | Modest | |
| SW948 | >10000 | 1.5 | 0.3 | 0.4 | 1.3 | 0.4 | No Synergy | No Synergy | |
| T84 | >10000 | 2.0 | 6.0 | 0.7 | 5004.7 | 0.6 | No Synergy | Modest | |

Table 2 Key:
Cell Line = Tumor-derived cell line
gIC₅₀ = Concentration of compound (nM) required to cause 50% growth inhibition
Yₘᵢₙ = The minimum cellular growth in the presence of Compound B (relative to DMSO control) as measured by % of that at T = 0 (number of cells at time of Compound B addition). A negative number indicates a net loss of cells relative to that at T = 0.
Yₘᵢₙ/T₀ = Yₘᵢₙ value divided by the T0 value whereas the Yₘᵢₙ is derived from the concentration-response curve and the T0 value represents the number of cells at the time of compound addition (CTG measurement).
EOHSA= Excess over highest single agent determination
BLISS = Bliss synergy determination
Comb Index = Combination Index score

Study #2: In Vitro Combination Studies of BRAF (Compound A) and PI3K Inhibitors (Compound B) on Cancer Cell Lines from Multiple Origins Encoding Different Mutations within the MAPK and AKT/PI3K Pathways
Drug combinations experiments were carried out in 384-well plates. Cell were plated in 384-well plates at 500 cells/well in culture media appropriate for each cell type, supplemented with 10% FBS and 1% penicillin/streptomycin, and incubated overnight at 37° C., 5% CO₂. Sixteen concentrations of 2 folds dilution of each drug were tested in matrix for cell growth inhibition. Concentrations tested for BRAF inhibitor Compound A were 10 μM-0.3 nM and for PI3K inhibitor (Compound B) were 5 μM-0.15 nM. Cells were treated with compound combination and incubated at 37° C. for 72 hours. Cell growth was measured using CellTiter-Glo® reagent according to the manufacturer's protocol and signals were read on a Perkin Elmer EnVision™ reader set for luminescence mode with a 0.5-second read. Results are expressed as a percentage inhibition compared to DMSO treated cells and background correction was made by subtraction of values from wells containing no cells.
For the purposes of this study the metric of Excess Over Highest Single Agent (EOHSA) was used to determine the degree of synergy between each compound.
A detailed description of EOHSA calculations can be found above. Briefly, the response (percent inhibition compared to untreated samples and normalized to media alone) of Compound A at “a” concentration (Rₐ) and that of Compound B at “b” concentration (R_{b}) is compared to response of the mixture of Compounds A & B at concentrations “a” and “b” respectively (R_{ab}). The equation:

R_{ab}>10% of the higher value among Rₐ and R_{b}=additive

R_{ab}<−10% of the higher value among Rₐ and R_{b}=antagonism

Using this formula, if R_{ab} is greater by 10% or more than the highest value between Rₐ and R_{b} the drug combination is considered ‘additive’. If R_{ab} is smaller by 10% or more than the highest value between Rₐ and R_{b} the drug combination is ‘antagonistic’. In this case, ‘additive’ cell lines are considered more synergistic than ‘antagonistic’ cell lines.
The number of combinations in the 16×16 matrix responding in an additive manner to the combination treatment were enumerated and summarized in Table 3. On this table we assigned a combination on a given cell line to be more beneficial (gray square)>20% (51 combination out of 256 tested) of combinations tested showed additivity as defined by a value greater than 10% Excess Over the Highest Single Agent (10% EOHSA).

TABLE 3
Combination effect of PI3K and BRAF inhibitor on multiple cancer cell lines.

| | | | | # of drug | % > |
|---|---|---|---|---|---|
| | | | | combinations | than |
| Origin | Cell Lines | MAPK | PI3K/PTEN | w EOHSA | EOHSA |
| Skin | A375P | BRAF^{V600E} | WT/WT | 101 | 39 |
| Colon | RKO | BRAF^{V600E} | H1047R/WT | 126 | 49 |
| Skin | A101D | BRAF^{V600E} | WT/G165_*404de | 68 | 27 |
| Skin | SK-MEL-5 | BRAF^{V600E} | WT/inc | 110 | 43 |
| | | NRAS^{G12V} | | | |
| Lung | A-549 | KRAS^{G12S} | WT/WT | 39 | 15 |
| Colon | LoVo | KRAS^{G13D} | WT/WT | 50 | 20 |
| Colon | HCT116 | KRAS^{G13D} | H1047R/WT | 51 | 20 |
| Skin | SK-MEL-2 | NRAS^{Q61R} | WT/WT | 62 | 24 |
| Lung | H1299 | NRAS^{Q61R} | WT/WT | 67 | 26 |
| Sarcoma | HT-1080 | NRAS^{Q61K} | WT/WT | 89 | 35 |
| Breast | MDA-MB-231 | NRAS^{Q61K} | WT/WT | 88 | 34 |

These data demonstrate that the combination of PI3K and BRAF inhibitors is favourable on multiple cancer cell lines from multiple origins independent of the mutational status of key oncogenes within the MAPK or the AKT/PI3K pathways as multiple drug combinations (>20%) showed inhibitory activity >10% Excess Over Highest Single Agent (EOHSA).
Study #3: In Vitro Cell Growth Inhibition by Compound A, Compound B, and their Combination in Tumor Cell Lines

### Methods:

### Cell Lines and Growth Conditions

Melanoma A375 PF11 line was derived from A375 (ATCC). 12R5-1, 12R5-3, 12R8-1, 12R8-3, 16R5-2, 16R6-3 and 16R6-4 are single cell clones derived from mixed populations of A375 PF11 cells that were selected to grow in Compound A to concentrations of 1200 and 1600 nM. All lines were cultured in RPMI 1640 medium containing 10% fetal bovine serum (FBS).

### Cell Growth Inhibition Assay and Combination Data Analysis

All cells were cultured for a minimum of 72 hours prior to cell plating. Cells were assayed in a 96-well tissue culture plate (NUNC 136102) of RPMI medium containing 10% FBS for all cells at 1,000 cells per well. Approximately 24 hours after plating, cells were exposed to ten, three-fold serial dilutions of compound or the combination of the two agents at a constant molar to molar ratio of 1:10 Compound A to Compound B in RPMI media containing 10% FBS. Cells were incubated in the presence of compounds for 3 days. ATP levels were determined by adding Cell Titer Glo® (Promega) according to the manufacturer's protocol. Briefly, Cell Titer Glo® was added each plate, incubated for 30 minutes then luminescent signal was read on the SpectraMax L plate reader with a 0.5 sec integration time.

Inhibition of cell growth was estimated after treatment with compound or combination of compounds for three days and comparing the signal to cells treated with vehicle (DMSO). Cell growth was calculated relative to vehicle (DMSO) treated control wells. Concentration of compound that inhibits 50% of control cell growth (IC₅₀) was interpolated when y=50% of the vehicle control using nonlinear regression with the equation, y=(A+(B−A)/(1+(C/x)̂D))), where A is the minimum response (yₘᵢₙ), B is the maximum response (yₘₐₓ), C is the inflection point of the curve (EC₅₀) and D is the Hill coefficient.

Combination effects on potency were evaluated using Combination Index (CI) which was calculated with the back-interpolated IC₅₀ values and the mutually non-exclusive equation derived by Chou and Talalay (Chou T C, Talalay P. Quantitative analysis of dose-effect relationships: the combined effects of multiple drugs or enzyme inhibitors. Adv Enzyme Regul 1984; 22:27-55.) A detailed description of the CI is found above. In general, a CI value <0.9, between 0.9 and 1.1, or >1.1 indicates synergy, additivity and antagonism, respectively. In general, the smaller the CI number, the greater is the strength of synergy.

The combination effects on the response scale were quantified by Excess Over Highest Single Agent (EOHSA) based on the concept of nonlinear blending as described in detail by Peterson and Novick (2007) and Peterson (2010) [Peterson J J, Novick S J. J Recept Signal Transduct Res 2007; 27(2-3):125-46, Peterson J. Frontiers of Bioscience S2, 483-503. 2010] EOHSA values are defined as increases in improvement (here, in ‘percentage points’ (ppts) difference) produced by the combination over the best single agent at its component dose level for the combination. Details on the calculation of EOHSA can be found above. For single agent and combination treatments, cells were exposed to compounds at a fixed-dose-ratio, and dose response curves were fit to the experimental data and analyzed using regression models. At specified total dose levels of IC₅₀ along the dose response curve, the dose combination (corresponding to IC₅₀) was determined for making EOHSA statistical inferences. More specifically, for a combination drug experiment involving drug 1 at dose d1 and drug 2 at dose d2, (i.e., total dose equals d1+d2) is said to have a positive EOHSA if the mean response at the combination is better than the mean response to drug 1 at dose d1 or drug 2 at dose d2.

### Results:

The effect of cell growth inhibition by a BRAF inhibitor Compound A, a PI3K inhibitor Compound B and their combination was determined in a panel of human melanoma cell lines. The mean IC₅₀s (from at least two independent experiments) and the combination effects at IC₅₀s are summarized in Table 4 with BRAF mutation status. A375 PF11 cells with BRAF V600E mutation were highly sensitive to either Compound A (IC₅₀=0.059 μM) or Compound B (IC₅₀=0.048 μM) single agent. The combination of Compound A and Compound B were synergistic demonstrated by a CI value of 0.74 in A375 PF11 cells. The seven Compound A resistant clones (12R8-3, 12R8-1, 12R5-3, 16R5-2, 16R6-3, 16R6-4 and 12R5-1 derived from the A375 PF11 melanoma cell line) displayed IC₅₀s ranging from 0.041 to 0.212 μM in response to Compound B alone, and responded to the combination of Compound A and Compound B with IC₅₀s ranging from 0.256-0.0.731 μM for Compound A and 0.026 to 0.073 μM for Compound B. The combination of Compound A and Compound B showed enhancement cell growth inhibition with EOHSA values from 3-34 ppts in the melanoma lines.

Table 4. Cell growth inhibition by Compound A, Compound B and their combination in human tumor cell lines.

TABLE 4

| | | | IC₅₀ values in micromolar (mean ± std) | | | | | |
|---|---|---|---|---|---|---|---|---|
| | | | | | Compound A or B = 10:1 | | Combination Effects at | |
| Tumor Cell | | Mutation Status | Single Agent | | molar ratio combination | | IC₅₀ | |
| Lines | | KRAS/BRAF | Compound A | Compound B | Compound A | Compound B | CI | EOHSA (ppt) |
| Melanoma | A375PF11 | BRAF_V600E | 0.059 ± 0.011 | 0.048 ± 0.009 | 0.044 ± 0.019 | 0.004 ± 0.002 | 0.74 ± 0.03 | 8 ± 2 |
| | 12R5-1 | BRAF_V600E | >10 | 0.212 ± 0.038 | 0.643 ± 0.227 | 0.064 ± 0.023 | N/A | 34 ± 1 |
| | 12R5-3 | BRAF_V600E | >10 | 0.088 ± 0.002 | 0.505 ± 0.085 | 0.050 ± 0.009 | N/A | 12 ± 2 |
| | 12R8-1 | BRAF_V600E | >10 | 0.103 ± 0.016 | 0.680 ± 0.182 | 0.068 ± 0.018 | N/A | 10 ± 2 |
| | 12R8-3 | BRAF_V600E | >10 | 0.129 ± 0.024 | 0.612 ± 0.008 | 0.061 ± 0.001 | N/A | 23 ± 4 |
| | 16R5-2 | BRAF_V600E | >10 | 0.041 ± 0.018 | 0.256 ± 0.057 | 0.026 ± 0.006 | N/A | 7 ± 3 |
| | 16R6-3 | BRAF_V600E | >10 | 0.080 ± 0.017 | 0.731 ± 0.204 | 0.073 ± 0.020 | N/A | 3 ± 3 |
| | 16R6-4 | BRAF_V600E | >10 | 0.061 ± 0.010 | 0.408 ± 0.031 | 0.041 ± 0.003 | N/A | 13 ± 6 |

Table 4 Key:
IC₅₀: the concentration of Compound as single agent, or the concentration of Compound A or B in combination when Compound A and Compound B = 10:1 molar ratio that reduces cell growth by 50%;
CI; Combination Index;
N/A = not applicable
EOHSA: Excess over Highest Single Agent, measured as a percentage.

### Example 1

### Capsule Composition

An oral dosage form for administering a combination of the present invention is produced by filing a standard two piece hard gelatin capsule with the ingredients in the proportions shown in Table I, below.

TABLE I

| INGREDIENTS | AMOUNTS |
|---|---|
| N-{3-[5-(2-amino-4-pyrimidinyl)-2-(1,1-dimethylethyl)-1,3- | 100 mg |
| thiazol-4-yl]-2-fluorophenyl}-2,6- | |
| difluorobenzenesulfonamide (Compound A) | |
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Mannitol | 250 mg |
| Talc | 50 mg |
| Magnesium Stearate | 20 mg |

### Example 2

### Capsule Composition

An oral dosage form for administering one of the compounds of the present invention is produced by filing a standard two piece hard gelatin capsule with the ingredients in the proportions shown in Table II, below.

TABLE II

| INGREDIENTS | AMOUNTS |
|---|---|
| N-{3-[5-(2-amino-4-pyrimidinyl)-2-(1,1-dimethylethyl)-1,3- | 100 mg |
| thiazol-4-yl]-2-fluorophenyl}-2,6- | |
| difluorobenzenesulfonamide (Compound A) | |
| Mannitol | 200 mg |
| Talc | 320 mg |
| Magnesium Stearate | 80 mg |

### Example 3

### Capsule Composition

An oral dosage form for administering one of the compounds of the present invention is produced by filing a standard two piece hard gelatin capsule with the ingredients in the proportions shown in Table III, below.

TABLE III

| INGREDIENTS | AMOUNTS |
|---|---|
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Mannitol | 50 mg |
| Talc | 25 mg |
| Magnesium Stearate | 2 mg |

### Example 4

### Tablet Composition

The sucrose, microcrystalline cellulose and the compounds of the invented combination, as shown in Table IV below, are mixed and granulated in the proportions shown with a 10% gelatin solution. The wet granules are screened, dried, mixed with the starch, talc and stearic acid, then screened and compressed into a tablet.

TABLE IV

| INGREDIENTS | AMOUNTS |
|---|---|
| N-{3-[5-(2-amino-4-pyrimidinyl)-2-(1,1-dimethylethyl)-1,3- | 100 mg |
| thiazol-4-yl]-2-fluorophenyl}-2,6- | |
| difluorobenzenesulfonamide (Compound A) | |
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Microcrystalline cellulose | 250 mg |
| sucrose | 50 mg |
| starch | 50 mg |
| talc | 20 mg |
| stearic acid | 2 mg |

### Example 5

### Tablet Composition

The sucrose, microcrystalline cellulose and one of the compounds of the invented combination, as shown in Table V below, are mixed and granulated in the proportions shown with a 10% gelatin solution. The wet granules are screened, dried, mixed with the starch, talc and stearic acid, then screened and compressed into a tablet.

TABLE V

| INGREDIENTS | AMOUNTS |
|---|---|
| N-{3-[5-(2-amino-4-pyrimidinyl)-2-(1,1-dimethylethyl)-1,3- | 100 mg |
| thiazol-4-yl]-2-fluorophenyl}-2,6- | |
| difluorobenzenesulfonamide (Compound A) | |
| Microcrystalline cellulose | 300 mg |
| Sucrose | 40 mg |
| Starch | 20 mg |
| Talc | 10 mg |
| stearic acid | 5 mg |

### Example 6

### Tablet Composition

The sucrose, microcrystalline cellulose and one of the compounds of the invented combination, as shown in Table VI below, are mixed and granulated in the proportions shown with a 10% gelatin solution. The wet granules are screened, dried, mixed with the starch, talc and stearic acid, then screened and compressed into a tablet.

TABLE VI

| INGREDIENTS | AMOUNTS |
|---|---|
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Microcrystalline cellulose | 60 mg |
| Sucrose | 5 mg |
| Starch | 10 mg |
| Talc | 5 mg |
| stearic acid | 2 mg |

While the preferred embodiments of the invention are illustrated by the above, it is to be understood that the invention is not limited to the precise instructions herein disclosed and that the right to all modifications coming within the scope of the following claims is reserved.

1. A combination comprising:
(i) a first compound of Structure (I): 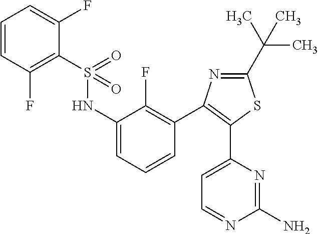
or a pharmaceutically acceptable salt or solvate thereof; and
(ii) a second compound which is a compound of Structure (II) 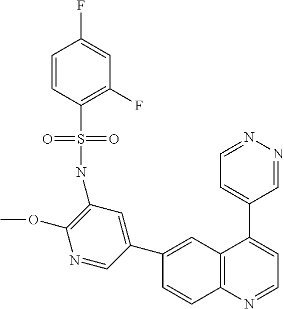
or a pharmaceutically acceptable salt thereof.

2. A combination according to claim 1 where the compound of Structure (I) is in the form of a methanesulfonate salt and the compound of Structure (II) is in the form of a free base.

3. A combination kit comprising a combination according to claim 1 together with a pharmaceutically acceptable carrier or carriers.

4. A combination according to claim 1 where the amount of the compound of Structure (I) is an amount selected from 10 mg to 300 mg, and that amount is administered from 1 to 4 times per day, and the amount of the compound of Structure (II) is an amount selected from 0.5 mg to 20 mg, and that amount is administered once per day.

5. A combination kit comprising a combination according to claim 1 together with a pharmaceutically acceptable carrier or carriers.

6. A method of treating cancer in a human in need thereof which comprises the in vivo administration of a therapeutically effective amount of a combination of N-{3-[5-(2-amino-4-pyrimidinyl)-2-(1,1-dimethylethyl)-1,3-thiazol-4-yl]-2-fluorophenyl}-2,6-difluorobenzenesulfonamide or a pharmaceutically acceptable salt thereof and 2,4-difluoro-N-{2-(methyloxy)-5-[4-(4-pyridazinyl)-6-quinolinyl]-3-pyridinyl}benzenesulfonamide, to such human,
wherein the combination is administered within a specified period, and
wherein the combination is administered for a duration of time.

7. A method of claim 6, which comprises the in vivo administration of a therapeutically effective amount of a combination of N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate salt and 2,4-difluoro-N-{2-(methyloxy)-5-[4-(4-pyridazinyl)-6-quinolinyl]-3-pyridinyl}benzenesulfonamide, to such human,
wherein the combination is administered within a specified period, and
wherein the combination is administered for a duration of time.

8. A method according to claim 6 wherein the amount of N-{3-[5-(2-amino-4-pyrimidinyl)-2-(1,1-dimethylethyl)-1,3-thiazol-4-yl]-2-fluorophenyl}-2,6-difluorobenzenesulfonamide or a pharmaceutically acceptable salt thereof, is selected from about 10 mg to about 300 mg, and that amount is administered from 1 to 3 times per day, and the amount of 2,4-difluoro-N-{2-(methyloxy)-5-[4-(4-pyridazinyl)-6-quinolinyl]-3-pyridinyl}benzenesulfonamide is selected from about 0.5 mg to about 10 mg.

9. A method according to claim 6 wherein the amount of N-{3-[5-(2-amino-4-pyrimidinyl)-2-(1,1-dimethylethyl)-1,3-thiazol-4-yl]-2-fluorophenyl}-2,6-difluorobenzenesulfonamide or a pharmaceutically acceptable salt thereof, is selected from about 70 mg to about 260 mg, and that amount is administered twice per day, and the amount of 2,4-difluoro-N-{2-(methyloxy)-5-[4-(4-pyridazinyl)-6-quinolinyl]-3-pyridinyl}benzenesulfonamide is selected from about 0.5 mg to about 6 mg

10. A method according to claim 8 wherein N-{3-[5-(2-amino-4-pyrimidinyl)-2-(1,1-dimethylethyl)-1,3-thiazol-4-yl]-2-fluorophenyl}-2,6-difluorobenzenesulfonamide or a pharmaceutically acceptable salt thereof, and 2,4-difluoro-N-{2-(methyloxy)-5-[4-(4-pyridazinyl)-6-quinolinyl]-3-pyridinyl}benzenesulfonamide are administered within 12 hours of each other each day for a period of at least 7 consecutive days, optionally followed by one of more repeating cycles.

11. A method according to claim 6 wherein N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate salt and the amount of 2,4-difluoro-N-{2-(methyloxy)-5-[4-(4-pyridazinyl)-6-quinolinyl]-3-pyridinyl}benzenesulfonamide are administered within 12 hours of each other each day for a period of at least 14 consecutive days, optionally followed by one of more repeating cycles.

12. A method of treating cancer in a human in need thereof which comprises one or more dosing cycles, wherein each said cycle comprises (1) administering to the human from about 10 to 300 mg of N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, 1-4 times a day for 1-30 days; and (2) periodically administering to the human from about 0.05 mg to 10 mg of 2,4-difluoro-N-{2-(methyloxy)-5-[4-(4-pyridazinyl)-6-quinolinyl]-3-pyridinyl}benzenesulfonamide, or a pharmaceutically acceptable salt or solvate.

13. (canceled)

14. A method of treating cancer in a human in need thereof which comprises one or more dosing cycles, wherein each said cycle comprises (1) administering to the human from about 0.05 to 10 mg of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, once or twice a day for 1-30 days; and (2) periodically administering to the human from about 10 to 300 mg of N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof for 1-30 days.

15. (canceled)

16. A method of claim 12, wherein 2,4-difluoro-N-{2-(methyloxy)-5-[4-(4-pyridazinyl)-6-quinolinyl]-3-pyridinyl}benzenesulfonamide is administered, once every 2-4 days.

17. A method of claim 12, wherein 2,4-difluoro-N-{2-(methyloxy)-5-[4-(4-pyridazinyl)-6-quinolinyl]-3-pyridinyl}benzenesulfonamide is administered, once every 5-7 days.

18. A method of claim 12, wherein 2,4-difluoro-N-{2-(methyloxy)-5-[4-(4-pyridazinyl)-6-quinolinyl]-3-pyridinyl}benzenesulfonamide is administered, once every 8-15 days.

19. A method of claim 14, wherein N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate is administered, once every 2-4 days.

20. A method of claim 14, wherein N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate is administered once every 5-7 days.

21. A method of claim 14, wherein N-{3-[5-(2-amino-4-pyrimidinyl)-2-(1,1-dimethylethyl)-1,3-thiazol-4-yl]-2-fluorophenyl}-2,6-difluorobenzenesulfonamide methanesulfonate is administered once every 8-15 days.

22. A method of treating cancer in a human in need thereof which comprises one or more repeating dosing cycles, wherein each said cycle comprises administering to the human from about 10 to 300 mg of N-{3-[5-(2-amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt or solvate thereof, 1-4 times a day for 5-14 days, followed by administering to the human from about 0.05 mg to 10 mg of 2,4-difluoro-N-{2-(methyloxy)-5-[4-(4-pyridazinyl)-6-quinolinyl]-3-pyridinyl}benzenesulfonamide, or a pharmaceutically acceptable salt or solvate for 5-14 days.

23. (canceled)

24. A method according to claim 12, wherein said cancer is melanoma or colon.

25. (canceled)

26. A combination according to claim 1 wherein said second compound is in the form of free base.

27.-30. (canceled)

31. A method of claim 8, wherein said cancer is melanoma, lung, pancreatic, breast or colon.

32.-33. (canceled)

34. A method according to claim 12, wherein said cancer is melanoma which has progressed after being treated with a BRaf inhibitor.

35. (canceled)

36. A method according to claim 12, wherein said cancer is colon cancer which has progressed after being treated with a BRaf inhibitor.

37. (canceled)
